(19) United States
(12) Patent Application Publication
(10) Pub. No.: US 2008/0032889 A1
(43) Pub. Date: Feb. 7, 2008

(54) 6-(2-Fluorophenyl)-Triazolopyrimidines, Method For The Production Thereof, Use Thereof For Controlling Harmful Fungi, And Agents Containing The Same

(75) Inventors: Carsten Blettner, Mannheim (DE); Markus Gewehr, Kastellaun (DE); Wassilios Grammenos, Ludwigshafen (DE); Thomas Grote, Wachenheim (DE); Udo Hunger, Mainz (DE); Bernd Muller, Frankenthal (DE); Matthias Niedenbruck, Limburgerhof (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Barbara Nave, Deidesheim (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF AKTIENGESELLSCHAFT, Ludwigshafen (DE)

(21) Appl. No.: 11/630,558

(22) PCT Filed: Jun. 14, 2005
(86) PCT No.: PCT/EP05/06342

§ 371 Date: Dec. 22, 2006

(30) Foreign Application Priority Data
Jun. 22, 2004 (DE) 10 2004 030 166.2

(51) Int. Cl. A01N 43/90; A01C 1/06; C07D 487/04

(52) U.S. Cl. 504/100; 514/259.31; 544/263

(57) ABSTRACT

Substituted triazolopyrimidines of the formula I 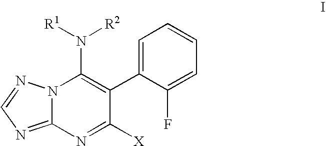 in which the substituents are as defined below:
R¹ is alkyl, halocycloalkyl, alkenyl, cycloalkyl, cycloalkenyl, halocycloalkenyl, alkynyl, haloalkynyl or naphthyl, or a five- or six-membered saturated, partially unsaturated or aromatic heterocycle comprising one to four heteroatoms from the group consisting of O, N and S,
R² is hydrogen, alkyl or one of the groups mentioned under R¹;
   R¹ and/or R² may carry one to four identical or different groups R^{a}:
   R^{a} is chlorine, bromine, iodine, cyano, nitro, hydroxyl, alkyl, alkylcarbonyl, cycloalkyl, alkoxy, haloalkoxy, alkoxycarbonyl, alkylthio, alkylamino, dialkyl-amino, alkenyl, cycloalkenyl, alkenyloxy, haloalkenyloxy, alkynyl, halo-alkynyl, alkynyloxy, haloalkynyloxy, cycloalkoxy, cycloalkenyloxy, oxyalkylenoxy, naphthyl, a five- to ten-membered saturated, partially unsaturated or aromatic heterocycle comprising one to four heteroatoms from the group consisting of O, N and S, where these aliphatic, alicyclic or aromatic groups for their part may be substituted according to the description; and X is halogen; processes for preparing these compounds, compositions comprising them and their use for controlling phytopathogenic harmful fungi.

The present invention relates to substituted triazolopyrimidines of the formula I

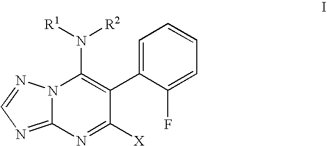

in which the substituents are as defined below:

R¹ is C₄-C₈-alkyl, C₃-C₈-halocycloalkyl, C₂-C₈-alkenyl, C₃-C₆-cycloalkenyl, C₃-C₆-halocycloalkenyl, C₂-C₈-alkynyl, C₂-C₈-haloalkynyl or naphthyl, or a five- or six-membered saturated, partially unsaturated or aromatic heterocycle comprising one to four heteroatoms from the group consisting of O, N and S,

R² is hydrogen, C₁-C₃-alkyl or one of the groups mentioned under R¹;
R¹ and/or R² may carry one to four identical or different groups R^{a}:
R^{a} is chlorine, bromine, iodine, cyano, nitro, hydroxyl, C₁-C₆-alkyl, C₁-C₆-alkyl-carbonyl, C₃-C₆-cycloalkyl, C₁-C₆-alkoxy, C₁-C₆-haloalkoxy, C₁-C₆-alkoxy-carbonyl, C₁-C₆-alkylthio, C₁-C₆-alkylamino, di-C₁-C₆-alkylamino, C₂-C₈-alkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C6-haloalkynyloxy, C₃-C₆-cycloalkoxy, C₃-C₆-cycloalkenyloxy, oxy-C₁-C₃-alkyleneoxy, naphthyl, a five- to ten-membered saturated, partially unsaturated or aromatic heterocycle comprising one to four heteroatoms from the group consisting of O, N and S,
   where these aliphatic, alicyclic or aromatic groups for their part may carry one to three groups R^{b}:
   R^{b} is chlorine, bromine, iodine, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxy-carbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the alkenyl or alkynyl groups mentioned in these radicals comprise 2 to 8 carbon atoms;
      and/or one to three of the following radicals:
      cycloalkyl, cycloalkoxy, heterocyclyl, heterocyclyloxy, where the cyclic systems comprise 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals comprise 5 or 6 ring members, where the cyclic systems may be partially or fully halogenated or substituted by alkyl or haloalkyl groups; and

X is halogen.

Moreover, the invention relates to processes for preparing these compounds, to compositions comprising them and to their use for controlling phytopathogenic harmful fungi.

Certain 5-halo-6-(2-fluorophenyl)-7-aminotriazolopyrimidines are known from EP-A 550 113, EP-A 989 130 and GB 23 55 261. It is known that these compounds are suitable for controlling harmful fungi.

The compounds according to the invention differ from those described in the above-mentioned publications by the properties of the substituents of the 7-amino group.

However, the action of the prior-art compounds is in many cases unsatisfactory. Accordingly, it is an object of the present invention to provide compounds having improved activity and/or a broader activity spectrum.

We have found that this object is achieved by the compounds defined at the outset. Furthermore, we have found processes and intermediates for their preparation, compositions comprising them and methods for controlling harmful fungi using the compounds I.

The compounds according to the invention can be obtained by different routes. Advantageously, they are prepared by reacting 5-aminotriazole of the formula II with appropriately substituted phenylmalonates of the formula III in which R is alkyl, preferably C₁-C₆-alkyl, in particular methyl or ethyl.

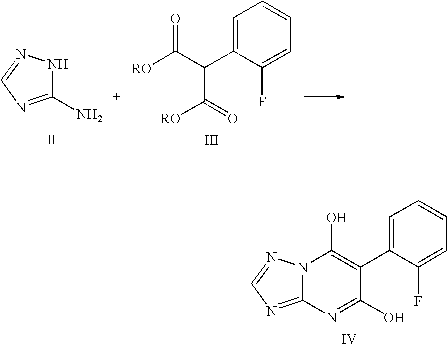

This reaction is usually carried out at temperatures of from 80° C. to 250° C., preferably from 120° C. to 180° C., in the absence of a solvent or in an inert organic solvent in the presence of a base [cf. EP-A 770 615] or in the presence of acetic acid under the conditions known from Adv. Het. Chem. 57 (1993), 81ff.

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethyl-acetamide. The reaction is particularly preferably carried out in the absence of a solvent or in chlorobenzene, xylene, dimethyl sulfoxide or N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines such as triisopropylethylamine, tributylamine, N-methylmorpholine or N-methylpiperidine.

The bases are generally employed in catalytic amounts; however, they can also be employed in equimolar amounts, in excess or, if appropriate, as solvents.

The starting materials are generally reacted with one another in equimolar amounts. In terms of yield, it may be advantageous to employ an excess of base and malonate III, based on the triazole.

Phenylmalonates of the formula III are advantageously obtained by reacting appropriately substituted bromobenzenes with dialkyl malonates under Cu(I) catalysis [cf. Chemistry Letters (1981), 367-370; EP-A 10 02 788].

The dihydroxytriazolopyrimidines of the formula IV are converted under the conditions known from WO-A 94/20501 into the dihalopyrimidines of the formula V in which Hal is a halogen atom, preferably a bromine or a chlorine atom, in particular a chlorine atom. Advantageous halogenating agents [HAL] are chlorinating agents or brominating agents, such as phosphorus oxybromide or phosphorus oxychloride, if appropriate in the presence of a solvent.

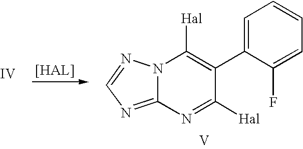

This reaction is usually carried out at from 0° C. to 150° C., preferably at from 80° C. to 125° C. [cf. EP-A 770 615].

Dihalopyrimidines of the formula V are reacted further with amines of the formula VI

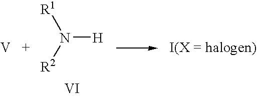

in which R¹ and R² are as defined in formula I, to give compounds of the formula I.

This reaction is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO-A 98/46608].

Preference is given to using a base, such as a tertiary amine, for example triethylamine, or an inorganic amine, such as potassium carbonate; it is also possible for excess amine of the formula VI to serve as base.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plants, or in the harmful fungus to be controlled.

In the definitions of the symbols given in the formulae above, collective terms were used which are generally representative of the following substituents:

halogen: fluorine, chlorine, bromine and iodine, in particular chlorine;

alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethyl-propyl, 1,2-dimethylpropyl, 1-methylpentyl, 2-methylpentyl, 3-methylpentyl, 4-methyl-pentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- to ten-membered saturated, partially unsaturated or aromatic heterocycle which comprises one to four heteroatoms from the group consisting of O, N and S:

5- or 6-membered heterocyclyl which comprises one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydro-thienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydro-pyrimidinyl, 5-hexahydropyrimidinyl and 2-piperazinyl;

5-membered heteroaryl which comprises one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;

6-membered heteroaryl which comprises one to three or one to four nitrogen atoms: 6-membered heteroaryl groups which, in addition to carbon atoms, may comprise one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The scope of the present invention includes the (R)- and (S)-isomers and the racemates of compounds of the formula I having chiral centers.

The particularly preferred embodiments of the intermediates with respect to the variables correspond to those of radicals L and R³ of the formula I.

With a view to the intended use of the triazolopyrimidines of the formula I, particular preference is given to the following meanings of the substituents, in each case on their own or in combination:

Particular preference is given to compounds I in which R¹ is C₄-C₈-alkyl or C₃-C₈-alkenyl.

Especially preferred are compounds I in which R² is hydrogen.

Preference is likewise given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R², preference is given to the (R)-configured isomers.

In addition, particular preference is also given to compounds of the formula I in which R¹ is CH(CH₃)—CH₂CH₃, CH(CH₃)—CH(CH₃)₂, CH(CH₃)—C(CH₃)₃, CH₂C(CH₃)═CH₂ or CH₂CH═CH₂ and R² is hydrogen or methyl.

A preferred embodiment of the invention relates to compounds of the formula I.1:

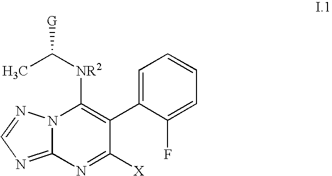

in which
G is C₂-C₆-alkyl, in particular ethyl, n- and isopropyl, n-, sec-, tert-butyl, and C₁-C₄-alkoxymethyl, in particular ethoxymethyl, or C₃-C₆-cycloalkyl, in particular cyclopentyl or cyclohexyl; and
R² is hydrogen or methyl.

Particular preference is given to compounds of the formula I.1 in which G is C₂-C₆-alkyl, in particular ethyl, n- and isopropyl, n-, sec-, tert-butyl and R² is hydrogen.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

Table 1

Compounds of the formula I in which X is chlorine and the combination of R¹ and R² for a compound corresponds in each case to one row of Table A

TABLE A

| No. | R¹ | R² |
|---|---|---|
| A-1 | CH₂CH₂CH₂CH₃ | H |
| A-2 | CH₂CH₂CH₂CH₃ | CH₃ |
| A-3 | CH₂CH₂CH₂CH₃ | CH₂CH₃ |
| A-4 | CH₂CH₂CH₂CH₃ | CH₂CH₂CH₃ |
| A-5 | CH₂CH₂CH₂CH₃ | CH₂CH₂CH₂CH₃ |
| A-6 | (±) CH(CH₃)—CH₂CH₃ | H |
| A-7 | (±) CH(CH₃)—CH₂CH₃ | CH₃ |
| A-8 | (±) CH(CH₃)—CH₂CH₃ | CH₂CH₃ |
| A-9 | (S) CH(CH₃)—CH₂CH₃ | H |
| A-10 | (S) CH(CH₃)—CH₂CH₃ | CH₃ |
| A-11 | (S) CH(CH₃)—CH₂CH₃ | CH₂CH₃ |
| A-12 | (R) CH(CH₃)—CH₂CH₃ | H |
| A-13 | (R) CH(CH₃)—CH₂CH₃ | CH₃ |
| A-14 | (R) CH(CH₃)—CH₂CH₃ | CH₂CH₃ |
| A-15 | (±) CH(CH₃)—CH(CH₃)₂ | H |
| A-16 | (±) CH(CH₃)—CH(CH₃)₂ | CH₃ |
| A-17 | (±) CH(CH₃)—CH(CH₃)₂ | CH₂CH₃ |
| A-18 | (S) CH(CH₃)—CH(CH₃)₂ | H |
| A-19 | (S) CH(CH₃)—CH(CH₃)₂ | CH₃ |
| A-20 | (S) CH(CH₃)—CH(CH₃)₂ | CH₂CH₃ |
| A-21 | (R) CH(CH₃)—CH(CH₃)₂ | H |
| A-22 | (R) CH(CH₃)—CH(CH₃)₂ | CH₃ |
| A-23 | (R) CH(CH₃)—CH(CH₃)₂ | CH₂CH₃ |
| A-24 | (±) CH(CH₃)—C(CH₃)₃ | H |
| A-25 | (±) CH(CH₃)—C(CH₃)₃ | CH₃ |
| A-26 | (±) CH(CH₃)—C(CH₃)₃ | CH₂CH₃ |
| A-27 | (S) CH(CH₃)—C(CH₃)₃ | H |
| A-28 | (S) CH(CH₃)—C(CH₃)₃ | CH₃ |
| A-29 | (S) CH(CH₃)—C(CH₃)₃ | CH₂CH₃ |
| A-30 | (R) CH(CH₃)—C(CH₃)₃ | H |
| A-31 | (R) CH(CH₃)—C(CH₃)₃ | CH₃ |
| A-32 | (R) CH(CH₃)—C(CH₃)₃ | CH₂CH₃ |
| A-33 | CH₂C(CH₃)═CH₂ | H |
| A-34 | CH₂C(CH₃)═CH₂ | CH₃ |
| A-35 | CH₂C(CH₃)═CH₂ | CH₂CH₃ |
| A-36 | CH₂CH═CH₂ | H |
| A-37 | CH₂CH═CH₂ | CH₃ |
| A-38 | CH₂CH═CH₂ | CH₂CH₃ |
| A-39 | CH(CH₃)CH═CH₂ | H |
| A-40 | CH(CH₃)CH═CH₂ | CH₃ |
| A-41 | CH(CH₃)CH═CH₂ | CH₂CH₃ |
| A-42 | CH(CH₃)C(CH₃)═CH₂ | H |
| A-43 | CH(CH₃)C(CH₃)═CH₂ | CH₃ |
| A-44 | CH(CH₃)C(CH₃)═CH₂ | CH₂CH₃ |
| A-45 | CH₂—C≡CH | H |
| A-46 | CH₂—C≡CH | CH₃ |
| A-47 | CH₂—C≡CH | CH₂CH₃ |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phakopsora pachyrhizi and P. meibomiae on soya,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg seed, preferably 1 to 200 g/100 kg, in particular 5 to 100 g/100 kg are generally used.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutyinaphthalene-sulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydro-naphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

2. Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinil,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafol, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, penthiopyrad, probenazole, proquinazid, pyrifenox, pyroquilon, quinoxyfen, silthiofam, SYP-Z048, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, mandipropamide, metrafenone, pencycuron, propamocarb, phosphorous acid, phthalide, tolclofos-methyl, quintozene or zoxamide,
strobilurins, such as azoxystrobin, dimoxystrobin, enestroburin (SYP-Z071), fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

With appropriate modification of the starting materials, the procedures given in the synthesis examples below were used to obtain further compounds I. The compounds obtained in this manner are listed in the table that follows, together with physical data.

### Example 1

### Preparation of 5-chloro-6-(2-fluorophenyl)-7-(3-methylbut-2-yl)-1,2,4-triazolo[1,5a]pyrimidine

0.15 g (0.53 mmol) 1. of 5,7-dichloro-6-(2-fluorophenyl)-1,2,4-triazolo[1,5a]pyrimidine (cf. WO 03/80615), 0.054 (0.54 mmol) of triethylamine and 0.047 g (0.54 mmol) of 3-methyl-2-butylamine in 1.5 ml of dichloromethane were stirred at 20-25° C. overnight. The reaction mixture was then washed with dil. hydrochloric acid and water, then dried and freed from the solvent. 0.11 g of the title compound remained as a lightly colored solid of m.p. 116-118° C.

¹H-NMR (CDCl₃, δ in ppm): 8.35 (s, 1H); 7.5 (m, 1H); 7.2-7.4 (m, 3H); 6.2 (s, broad, 1H); 3.1 (s, broad, 1H); 1.6 (m, broad, 1H); 1.0; 1.1 (2 d, 3H); 0.8 (2 d, 6H).

TABLE I

| | | | | phys. data |
|---|---|---|---|---|
| No. | R¹ | R² | X | (m.p. [° C.]) |
| I-1 | CH₂C(═CH₂)CH₃ | C₂H₅ | Cl | 106-108 |
| I-2 | CH₂CH═C(CH₃)₂ | CH₂—CH═C(CH₃)₂ | Cl | resin |
| I-3 | CH₂CH═C(CH₃)₂ | H | Cl | resin |
| I-4 | CH₂CH═CH₂ | C₂H₅ | Cl | 113-114 |
| I-5 | CH₂CH(CH₃)₂ | H | Cl | 132 |
| I-6 | CH₂CH(CH₃)₂ | CH₂—CH═CH₂ | Cl | 137 |
| I-7 | CH₂CH(CH₃)₂ | CH₃ | Cl | 143 |
| I-8 | CH₂CH(CH₃)₂ | C₂H₅ | Cl | 92 |
| I-9 | CH(CH₃)CH₂CH₃ | CH₃ | Cl | 141 |
| I-10 | CH(CH₃)CH(CH₃)₂ | CH₃ | Cl | 156 |
| I-11 | CH(CH₃)CH₂CH₂CH₃ | H | Cl | 88-90 |
| I-12 | CH(CH₃)(CH₂)₂CH(CH₃)₂ | H | Cl | resin |
| I-13 | CH₂CH₂CH(CH₃)₂ | CH₃ | Cl | 92 |
| I-14 | CH(CH₃)C(CH₃)₃ | H | Cl | 135-137 |
| I-15 | (R) CH(CH₃)C(CH₃)₃ | H | Cl | 125-127 |
| I-16 | CH(CH₃)CH(CH₃)₂ | H | Cl | 116-118 |
| I-17 | (R) CH(CH₃)CH(CH₃)₂ | H | Cl | 121-123 |
| I-18 | CH(CH₃)CH₂CH₃ | H | Cl | 123-125 |
| I-19 | (R) CH(CH₃)CH₂CH₃ | H | Cl | 121-132 |

Examples for the Action Against Harmful Fungi

The fungicidal action of the compounds of the formula I was demonstrated by the following tests:

The active compounds were formulated as a stock solution with 0.25% by weight of active compound in acetone or DMSO. 1% by weight of the emulsifier Uniperol® EL (wetting agent having emulsifying and dispersing action based on ethoxylated alkylphenols) was added to this solution, and the mixture was diluted with water to the desired concentration.

The comparative active compound used was the compound A known from EP-A 550 113 as example No. 41 of table 1:

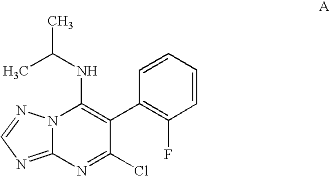

Comparative Experiment 1—Activity Against Gray Mold Caused by Botrytis cinerea, Protective Application

Potted bell pepper plants of the cultivar “California Wonder” were sprayed to runoff point with an aqueous suspension having the active compound concentration stated below. The suspension or emulsion had been prepared by dilution with water from a stock solution comprising 5% of active compound, 94% of cyclohexanone and 1% of emulsifier (Tween 20). 2-5 hours after the spray coating had dried on, the plants were dusted with spores of gray mold (Botrytis cinerea). The test plants were then incubated in a humid chamber at temperatures between 20 and 22° C. and at 100% relative atmospheric humidity. After about 3 days, the extent of the pathogen development was determined visually in % infection of the entire leaf area.

In this test, the plants which had been treated with 200 ppm of the compounds I-5, I-14 or I-18 showed an infection of 3 to 20%, whereas the plants which had been treated with 200 ppm of the comparative compound A and the untreated plants were 90% and 100% infected, respectively.

### Use Example 2

### Activity Against Alternaria solani on Tomatoes (Protective)

Leaves of tomato plants of the cultivar “Pixie II” which had been cultivated in pots up to the 4-leaf stage were sprayed to runoff point with an aqueous preparation of active compound which had been prepared from a stock solution of 5% active compound, 94% acetone and 1% emulsifier (Tween 20). After the spray coating had dried on (3-5 hours), the leaves were inoculated with an aqueous spore suspension of Alternaria solani (density 15×10³ spores per ml). The test plants were then placed in climatized chambers at 22 to 24° C. and 96 to 99% relative atmospheric humidity for 36 hours and then cultivated in a greenhouse at 21 to 23° C. and approximately 95% relative atmospheric humidity for a further 2 to 3 days. The extent of the development of the infection on the leaves was then determined visually.

In this test, the plants which had been treated with 200 ppm of the active compound I-3, I-4, I-5 or I-11 showed no or at most 3% infection, whereas the untreated plants were 90% infected.

### Use Example 3

### Activity Against Early Blight of Tomato Caused by Alternaria solani

Leaves of potted plants of the cultivar “Goldene Prinzessin” were sprayed to runoff point with an aqueous suspension having the concentration of active compound stated below. The next day, the leaves were infected with an aqueous spore suspension of Alternaria solani in a 2% strength biomalt solution having a density of 0.17×10⁶ spores/ml. The plants were then placed in a water-vapor-saturated chamber at temperatures between 20 and 22° C. After 5 days, the disease on the untreated but infected control plants had developed to such an extent that the infection could be determined visually in %.

In this test, the plants which had been treated with in each case 250 ppm of the active compounds I-14 to I-19 showed no infection, whereas the untreated plants were 100% infected.

1. A triazolopyrimidine of the formula I 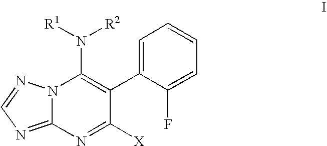
in which the substituents are as defined below:
R¹ is C₄-C₈-alkyl, C₃-C₈-halocycloalkyl, C₂-C₈-alkenyl, C₃-C₆-cycloalkenyl, C₃-C₆-halocycloalkenyl, C₂-C₈-alkynyl, C₂-C₈-haloalkynyl or naphthyl, or a five- or six-membered saturated, partially unsaturated or aromatic heterocycle comprising one to four heteroatoms from the group consisting of O, N and S,
R² is hydrogen, C₁-C₃-alkyl or one of the groups mentioned under R¹;
R¹ and/or R² may carry one to four identical or different groups R^{a}:
R^{a} is chlorine, bromine, iodine, cyano, nitro, hydroxyl, C₁-C₆-alkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, naphthyl, a five- to ten-membered saturated, partially unsaturated or aromatic heterocycle comprising one to four heteroatoms from the group consisting of O, N and S,
where these aliphatic, alicyclic or aromatic groups for their part may carry one to three groups R^{b}:
R^{b} is chlorine, bromine, iodine, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the alkenyl or alkynyl groups mentioned in these radicals comprise 2 to 8 carbon atoms;
and/or one to three of the following radicals:
cycloalkyl, cycloalkoxy, heterocyclyl, heterocyclyloxy, where the cyclic systems comprise 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals comprise 5 or 6 ring members, where the cyclic systems may be partially or fully halogenated or substituted by alkyl or haloalkyl groups; and
X is halogen.

2. The compound of the formula I according to claim 1 in which X is chlorine.

3. The compound according to claim 1, which corresponds to the formula I.1 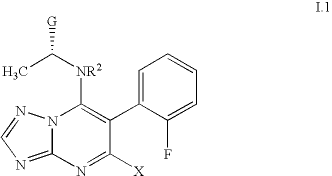
in which
G is C₂-C₆-alkyl, C₁-C₄-alkoxymethyl or C₃-C₆-cycloalkyl;
R² is hydrogen or methyl; and
X is halogen.

4. A process for preparing compounds of the formula I according to claim 1 by reacting 5-aminotriazole of the formula II 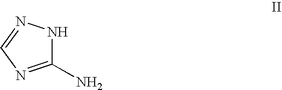
with phenylmalonates of the formula III, 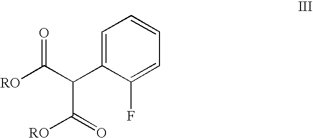
in which R is alkyl, to give dihydroxytriazolopyrimidines of the formula IV, 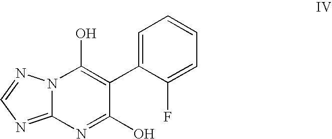
halogenation to give the dihalo compounds of the formula V, 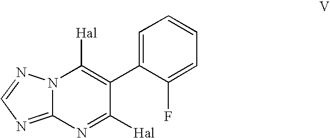
and reaction of V with amines of the formula VI 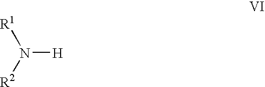
to give compounds of the formula I.

5. A fungicidal composition comprising a solid or liquid carrier and a compound of the formula I according to claim 1.

6. The fungicidal composition according to claim 5 comprising a further fungicidally active compound.

7. A method for controlling phytopathogenic harmful fungi, which method comprises treating the fungi or the materials, plants, the soil or seed to be protected against fungal attack with an effective amount of a compound of the formula I according to claim 1.

8. The method according to claim 7 wherein between 0.01 and 2.0 kg of active compound are applied per ha.

9. The method according to claim 7 wherein from 1 to 1000 g/100 kg of seed are applied.

10. Seed comprising from 1 to 1000 g of a compound of the formula I according to claim 1 per 100 kg.

11. The compound according to claim 2, which corresponds to the formula I.1 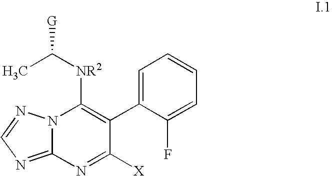
in which
G is C₂-C₆-alkyl, C₁-C₄-alkoxymethyl or C₃-C₆-cycloalkyl;
R² is hydrogen or methyl; and
X is halogen.

12. A process for preparing compounds of the formula I according to claim 2 by reacting 5-aminotriazole of the formula II 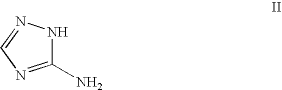
with phenylmalonates of the formula III, 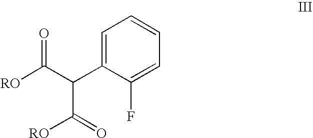
in which R is alkyl, to give dihydroxytriazolopyrimidines of the formula IV, 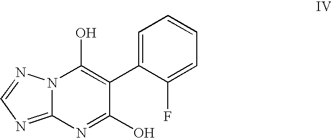
halogenation to give the dihalo compounds of the formula V, 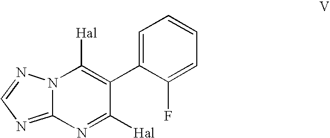
and reaction of V with amines of the formula VI 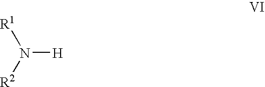
to give compounds of the formula I.

13. A process for preparing compounds of the formula I according to claim 3 by reacting 5-aminotriazole of the formula II 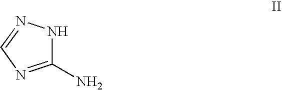
with phenylmalonates of the formula III, 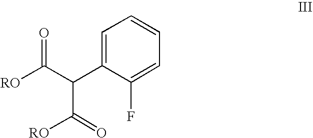
in which R is alkyl, to give dihydroxytriazolopyrimidines of the formula IV, 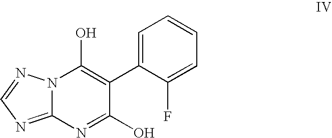
halogenation to give the dihalo compounds of the formula V, 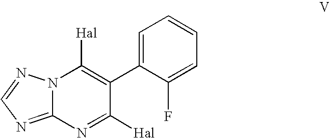
and reaction of V with amines of the formula VI 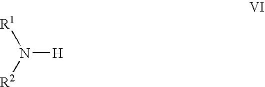
to give compounds of the formula I.
